(19) United States
(12) Patent Application Publication
(10) Pub. No.: US 2007/0128122 A1
(43) Pub. Date: Jun. 7, 2007

(54) Volatile material-containing compositions having an end of service indicator

(76) Inventors: Jonathan Robert Cetti, Mason, OH (US); Ross Howard Holthouse, Mason, OH (US); Mario Alonso, Loveland, OH (US); Zaiyou Liu, West Chester, OH (US); Fernando Ray Tollens, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/177,828

(22) Filed: Dec. 1, 2005

(51) Int. Cl. A61K 9/12; A01N 25/00; A61K 31/015

(52) U.S. Cl. 424/45; 514/766; 424/405

(57) ABSTRACT

Volatile material-containing compositions having an end of service indicator and methods of indicating the end of service of a volatile material emitted from a volatile material-containing composition are disclosed.

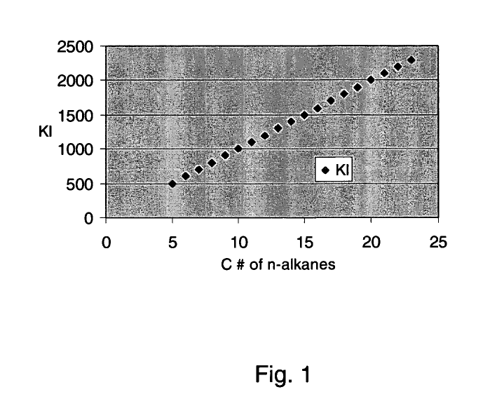

### FIELD OF THE INVENTION

The present invention relates to volatile material-containing compositions having an end of service indicator and methods of indicating the end of service of a volatile material emitted from a volatile material-containing composition.

### BACKGROUND OF THE INVENTION

Volatile material-containing compositions are used for various purposes. Such purposes include, but are not limited to releasing into a room or other space, volatile materials such as perfumes or scented materials, insecticides, air fresheners, deodorants, aromacology, aromatherapy, or any other volatile that acts to condition, modify, or otherwise charge the atmosphere or to modify the environment.

There are several drawbacks to known compositions used for these purposes. One significant drawback is the fact that it can be difficult for a user to determine when the volatile material has been depleted. For the purpose of this application, we will refer to the situation where a volatile has been depleted to the point of no longer providing the benefit intended by the manufacturer as “end of service.” Determining end of service can be difficult because the carrier of the volatile may still be present, thus leaving no visual indication that the volatile material has been depleted. This situation is complicated when multiple perfume ingredients are involved. Commonly, the more highly volatile perfume ingredients (referred to as the “top notes” and the “middle notes”) are depleted before the less volatile bottom notes. This results in a confusing situation for the user, since the device still emits a scent, but the scent character is different (predominately “bottom notes”). In this case, the user may not have a visual or clear olfactory indication of end of service, since a scent is still emitted. Unfortunately, a predominately “bottom note” scent is usually not as desirable as the full perfume profile. Often, the user would prefer to replace the volatile-containing composition at this point. Therefore, a need still exists for means to visually indicate the end of service of a volatile-containing material.

### SUMMARY OF THE INVENTION

This invention relates to volatile material-containing compositions having an end of service indicator and methods of indicating the end of service of a volatile material emitted from a volatile material-containing composition. Several non-limiting embodiments are described herein, each of which may constitute an invention in its own right or together with other components. In one non-limiting embodiment, the volatile material-containing composition comprises a carrier, at least one volatile material, and at least one volatile dye. The composition has a first state when energy is not applied to the composition, and a second energized state when energy is applied to the composition. The volatile material and volatile dye are emitted at a first level from the volatile material-containing composition in the first state and the volatile material and volatile dye are emitted from the volatile material-containing composition at a second higher level in the second state. The volatile material-containing composition returns to the first state when energy is no longer applied to the volatile material-containing composition. Preferably, the composition releases less than 10 mg/hour of the volatile material and less than 10 μg/hour of the volatile dye at 25° C. and 50% relative humidity (RH).

Methods of providing a visual indication of the depletion of a volatile material from a volatile material-containing composition are also disclosed.

Numerous other embodiments are also possible, including, but not limited to those described in the following detailed description.

### DETAILED DESCRIPTION OF THE INVENTION

This invention relates to volatile material-containing compositions having an end of service indicator and methods of indicating the end of service of a volatile material emitted from a volatile material-containing composition. Several non-limiting embodiments are described herein, as are several components of the system, each of which may constitute an invention in its own right or together with other components.

The volatile materials can be emitted in various facilities, which include but are not limited to rooms, houses, hospitals, offices, theaters, buildings, and the like, or into various vehicles such as trains, subways, automobiles, airplanes and the like.

The term “volatile materials” as used herein, refers to a material that is vaporizable. The terms “volatile materials”, “aroma”, and “scents”, as used herein, include, but are not limited to pleasant or savory smells, and, thus, also encompass scents that function as insecticides, air fresheners, deodorants, aromacology, aromatherapy, or any other volatile that acts to condition, modify, or otherwise charge the atmosphere or to modify the environment. It should be understood, however, that perfumes, aromatic materials, and scents will often be comprised of one or more volatile materials (which may form a unique and/or discrete unit comprised of a collection of volatile materials).

The term “volatile dyes,” as used herein, refers to soluble or insoluble coloring matter that is vaporizable. The chemical composition can be a single component or mixture. Preferably, the volatile dye is readily vaporizable at a relatively low temperature. For example, it is preferable that the volatile dye have a vapor pressure at room temperature greater than 1×10⁻⁵ torr. Preferably, the volatile dye has a Kovat's index (as determined by a DB-5 column) of from about 1000 to about 2000. More, preferably, the volatile dye has a Kovat's index of from about 1000 to about 1700.

Kovat's Index (KI, or Retention Index) is defined by the selective retention of solutes or perfume raw materials (PRMs) onto the chromatographic columns. It is primarily determined by the column stationary phase and the properties of solutes or PRMs. For a given column system, a PRM's polarity, molecular weight, vapor pressure, boiling point and the stationary phase property determine the extent of retention. To systematically express the retention of analyte on a given GC column, a measure called Kovat's Index (or retention index) is defined. Kovat's Index (KI) places the volatility attributes of an analyte (or PRM) on a column in relation to the volatility characteristics of n-alkane series on that column. Typical columns used are DB-5 and DB-1.

With this definition, the Kovat's index of a PRM, x, eluting at time t′, between two n-alkanes with number of carbon atoms n and N having corrected retention times t′ₙ and t′_{N} respectively will then be calculated as:

$$KI = 100 ( n + \frac{log {t}_{x}^{′} - log {t}_{n}^{′}}{log {t}_{N}^{′} - log {t}_{n}^{′}} ) \qquad ( 1 )$$

On a non-polar to slightly polar GC stationary phases, KI of PRMs are correlated with their relative volatility. For example, PRMs with smaller KI tends to be more volatile than that with larger KI. Ranking PRMs with their corresponding KI values give a good comparison of PRM evaporation rates in liquid-gas partitioning systems.

The volatile dye provides a visual indication of end of service by evaporating at a correlating rate to one or more of the volatile materials. The evaporation of the dye results in a color change in the volatile material-containing composition. For example, in a preferred embodiment the volatile material-containing composition is initially blue in color. As the composition is used, the volatile dye will be depleted, resulting in a reduction in the blue color. Depending on the carrier and other factors, end of service may be indicated by a complete loss of color or reduced shade of color. In a preferred embodiment, the volatile material-containing composition is contained in a device that has a color code or color guide to assist a user of the device in interpreting color changes in the composition.

The type of volatile dye and the amount used in the composition will vary depending on the desired indication. For example, in one embodiment, the depletion of the volatile dye will correlate with the depletion of the “middle note” perfume ingredient of the volatile material. In another embodiment, the depletion of the volatile dye will correlate with the depletion of the “top note” perfume ingredient of the volatile material. In an alternative embodiment, the depletion of the volatile dye will correlate with the depletion of a volatile material that is not a perfume.

In a preferred embodiment, the volatile dye is based off of the azulene (Bicyclo (5.3.0) Decapentaene) structure. An “azulene base structure” is a 10 carbon structure wherein a seven member ring is fused to a five member ring. Preferably, the volatile dye is selected from the group consisting of 6-methyl-azulene; 1-(1-azulenyl)-2,2,2-trifluoro-ethanone; 4,6,8-trimethyl-azulene; 7-ethyl-1,4-dimethyl-azulene; 1,4-dimethyl-7-(1-methylethyl)-azulene; azulene, and mixtures thereof. More preferably, the volatile dye is selected from the group consisting of 1,4-dimethyl-7-(1-methylethyl)-azulene, azulene, and mixtures thereof.

In one non-limiting embodiment, the volatile material-containing composition comprises a carrier, at least one volatile material, and at least one volatile dye. The composition has a first state when energy is not applied to the composition, and a second energized state when energy is applied to the composition. The volatile material and volatile dye are emitted at a first level from the volatile material-containing composition in the first state and the volatile material and volatile dye are emitted from the volatile material-containing composition at a second higher level in the second state. The volatile material-containing composition returns to the first state when energy is no longer applied to the volatile material-containing composition.

Preferably, the composition releases less than 10 mg/hour of the volatile material and less than 10 μg/hour of the volatile dye at 25° C. and 50% relative humidity (RH). More preferably, the composition releases less than 5 mg/hour of the volatile material and less than 5 μg/hour of the volatile dye at 25° C. and 50% relative humidity (RH). Even more preferably, the composition releases less than 1 mg/hour of the volatile material and less than 1 μg/hour of the volatile dye at 25° C. and 50% relative humidity (RH).

Preferably, the composition contains up to about 0.1% of volatile dye by weight. More preferably, the composition contains up to about 0.08% of volatile dye by weight. Preferably, the composition contains at least about 0.001% of volatile dye by weight. More preferably, the composition contains at least about 0.003% of volatile dye by weight.

In one embodiment, a system for dispensing scents into the environment can be provided which comprises one or more components containing one or more scents or aromatic materials. In such an embodiment, the system preferably comprises a dispensing device, such as a device and one or more aromatic material-containing articles of manufacture, or “scent-containing articles of manufacture”, which may be provided in the form of fragrance “cartridges”. Each cartridge can provide a single volatile composition, or a combination of different volatile materials, such as a combination of different scented materials. In certain embodiments, each of the cartridges provides a collection of scents that conveys, e.g., a theme, an experience, a physiological effect, and/or a therapeutic effect.

The volatile compositions of interest herein can be provided in any suitable form. In some embodiments, scents are provided by volatile compositions comprising perfume, such as perfume oils, that are incorporated onto or into a suitable carrier. The carriers can be provided in the following non-limiting forms: a solid, a liquid, a paste, a gel, beads, encapsulates, wicks, a carrier material, such as a porous material impregnated with or containing the perfume, and combinations thereof. In some embodiments, the carrier is in the form of a pliable solid which can be melted and have the perfume ingredients added thereto in order to form a composition that is in the form of a pliable solid structure or matrix at room temperature (73° F. (25° C.), 50% RH).

In certain embodiments, the volatile composition has a viscosity of from about 1,000 Cps to about 1,000,000 Cps, or more, measured at a shear stress of 100 Pa in a rotational rheometer, like the AR2000 (TA instruments New Castle, Del., USA), using a 40-mm diameter cone-and-plate geometry at 25° C. Such a composition can exist as a gel up to at least about 13,000 Cps. In certain embodiments when the composition is in the form of a pliable solid, it can have a viscosity of from about 100,000 to about 1,000,000 Cps.

In one non-limiting embodiment, at room temperature, the composition is in the form of a structure that is a structured polymeric pliable solid. Such a structure may be porous or non-porous. The structure may be homogeneous (which may also be referred to herein as “continuous”), or non-homogeneous. In many embodiments, it is desirable for the structure to be permeable to volatile materials contained therein. This will allow the structure to release the volatile materials contained therein when desired. In preferred versions of such an embodiment, the composition comprises a non-porous, homogeneous, permeable, structured polymeric pliable solid.

The volatile composition can be formed in a number of different manners. In one embodiment, the composition can be made by adding the volatile ingredient(s) and volatile dye(s) to a carrier, such as polyethylene glycol (or “PEG”). The volatile ingredients, such as perfumes, and the volatile dyes are preferably miscible with the carrier, and after cooling, forms a pliable solid-like at room temperature. PEG is available in various molecular weights. While PEG's having low molecular weights (or “MW”) (e.g., molecular weights less than 400) can be used as solvents for perfumes, such PEG's are liquids at room temperature, and may be used, but are not preferred for use in the compositions described herein. In more preferred embodiments of the composition, the MW of PEG is greater than or equal to about 1,000, or greater than or equal to about 4,000. It is desirable that the MW of PEG be greater than or equal to about 8,000. The molecular weight of PEG may be as high as 24,000, or higher. All molecular weights specified herein are weight average molecular weights.

Other suitable carriers are hydrogenated castor oil and high chain fatty acids, particularly those with a chain length of greater than or equal to 14 carbon atoms. In certain embodiments, it is desirable for the majority of the composition to comprise such a carrier and the volatile ingredient(s). Thus, such a carrier and the volatile ingredient(s) may comprise more than about 20%, alternatively, more than about 50% of the composition, by weight. In certain embodiments, it may be desirable for the composition (and/or the carrier) to also be substantially free of HPC (hydroxy propyl cellulose).

It may be desirable to utilize a structurant with the carrier. A structurant can be used for any suitable purpose. Examples of such purposes include, but are not limited to providing the structure formed by the composition with greater stability. The structurant can reduce the tendency of the structure to release the volatile material(s) and volatile dye(s) at low temperatures (e.g., ambient or storage or shipping temperatures). Thus, the volatile material(s) and volatile dye(s) will not be released until energy is applied to the structure in order to release the volatile material(s) and volatile dye(s). Any suitable structurant can be used. Suitable structurants comprise any substance that includes a divalent cation. Substances that comprise divalent cations include, but are not limited to magnesium and calcium containing molecules such as magnesium and calcium chloride, magnesium and calcium carbonate. Other suitable structurants include, but are not limited to derivatives of castor oil, including, but not limited to hydrogenated castor oil.

It may also be desirable for the composition to include at least one wax. Waxes can be used for any suitable purpose, including, but not limited to raising the melting temperature of structure formed by the composition for improved stability. Any suitable wax(es) can be used. In certain embodiments, it is desirable for the wax to have a melting point that is greater than that of the carrier. If the carrier is PEG, the melting point of the wax may, for example, be greater than about 50° C. Suitable waxes include, but are not limited to waxes that are derivatives of the carrier, for example, derivatives of PEG. Waxes that are derivatives of the carrier may be preferred because the structurants that are capable of structuring the carrier will also be able to structure the waxes in order to further raise the melting point of the entire matrix. It may also be desirable that the wax does not have an affinity for the volatile material so that it does not affect the emission rate or delivery of the volatile material.

In one embodiment, the composition is formed by combining polyethylene glycol (or “PEG”), hydrogenated castor oil, and a low level of at least one wax, at least one volatile ingredient, and at least one volatile dye.

The volatile ingredient(s) can comprise a number of components or compositions, including, but not limited to: fragrances (or perfume oils), flavors, pesticides, repellants, or mixtures thereof.

The volatile ingredient(s) and volatile dye(s) can be combined with the carrier material in any suitable manner. Several suitable manners in which the volatile ingredient(s) and volatile dye(s) can be combined with the carrier material include, but are not limited to: by entrapment; the volatile ingredient(s) and volatile dye(s) can be dissolved in the carrier material; the volatile ingredient(s) and volatile dye(s) can be partially encapsulated or completely encapsulated in the carrier material.

The components of the composition can be incorporated into the composition in any suitable amounts. In some embodiments, it may be desirable for the concentration of the volatile material(s) to be greater than about 5% of the composition. More preferably, the concentration of the volatile material(s) is greater than about 10% of the composition. In some embodiments, the concentration of the volatile material(s), such as the perfume ingredients, may be as high as about 75%, or more of the composition. In other embodiments, the amount of volatile material(s) may range from about 25% to about 75% of the composition. In some embodiments, the composition contains up to about 0.1% of volatile dye by weight. In other embodiments, the composition contains up to about 0.08% of volatile dye by weight. In some embodiments, the composition contains at least about 0.001% of volatile dye by weight. In other embodiments, the composition contains at least about 0.003% of volatile dye by weight. The carrier (such as polyethylene glycol) may comprise the balance of the composition. In some embodiments, the carrier may range from about 25% to about 75%, or more. In alternative embodiments, the carrier may be present in an amount that is less than this range. The structurant (such as hydrogenated castor oil) level may range from about 0 to about 15%, 20%, 30%, 40%, or more. The wax level may range from about 0 to about 3%, 5%, or more. All percentages stated herein are by weight of the composition, unless stated otherwise. The amounts of the components are typically selected so that they total 100%. However, it is also possible for other components to be added to the composition, in which case the weights of the components such as the carrier, volatile material(s), volatile dye(s), structurant, and wax may total less than 100% of the composition.

The structure (or matrix) comprising the composition can be thermally triggered or otherwise energized to emit the volatile material(s) and volatile dye(s). Such a structure can undergo a transition between a variety of different states depending on the temperature to which the structure is heated. For instance, in some embodiments, the composition can exist in any of the following phases: solid, gel, liquid, and mixtures thereof. Each phase of the composition can provide different volatilization characteristics. In the case of scented materials, this can include different volatilization rates, intensities, scent characters, emission profiles, etc. In some embodiments, the change in state of the composition is reversible in that it can change back to, or toward, more solid states. In some embodiments, it may be possible to vary the form or state of the composition from solid-like to gel-like by controlling the proportions of the components of the composition. For example, the composition will become less solid-like and more gel-like with the addition of additional structurant, such as hydrogenated castor oil. The reversible liquefication/gellation/solidification of the structure can be used to regulate or control the release of the volatile material. In most compositions, in the case of fragrance compositions, at lower temperatures, the more highly volatile perfume components (the “top notes”) will volatilize first. In the case of certain embodiments of the compositions described herein, if the composition is heated above its melting point (until it becomes a liquid), the perception of the volatile composition will be more true to the desired essence of the character, scent, flavor, etc. of the volatile material since all of the components of the material will be emitted at the same intensity at the desired temperature and time from the highly volatile perfume components (the “top notes”) to the less volatile (“bottom notes”). Thus, in certain embodiments, there is minimum partitioning of the volatile material composition and consistency of character/concentration over time. In the case of the examples set out herein, the melting point of the matrix is about 52° C. When energy is no longer applied, the structure goes back to a wax-like solid state or pliable solid which reduces the tendency of the volatile material to escape.

In certain embodiments, it is desirable for the composition to be heated to a temperature that is in excess of the melting point of the carrier. The addition of perfume ingredients will typically lower the melting temperature of the composition. As perfume ingredients are volatilized, the melting temperature of the remaining portion of the composition will increase. If the composition is always heated to a melting temperature above that of the carrier, then this will always provide sufficient energy to the composition in order to emit the volatile components therefrom.

The composition may provide certain advantages. It should be understood in this regard, however, that the composition need not provide any of these advantages unless specified in the appended claims. In some embodiments in the case of fragrance compositions, the composition can deliver a longer lasting aroma. For example, certain gels which have been previously used to contain volatile materials will release the more volatile perfume components even without being heated, or otherwise energized. This will reduce the longevity of such compositions, and will effect the character of the perfume that is emitted when the composition is heated. In some embodiments, the composition can retain the volatile material(s) better than some other compositions during periods when the volatile material(s) are not intended to be emitted. In some embodiments, the composition can be more compatible with the material of the container in which is placed (which may be referred to as “supporting material”). Often perfume oils are not compatible with plastics. However, when perfume oils are incorporated into the composition described herein, the composition may be more compatible with plastic materials. Without wishing to be bound to any particular theory, it is believed that the volatile material-containing composition described herein will have a greater surface tension than that of the perfume oil, to reduce or eliminate migration of the perfume oil from the composition, a phenomenon known as wicking. In some embodiments, the composition will have a surface tension of higher than 20 dyne/cm and lower than 25 dyne/cm. In some embodiments, the composition will have good stability at elevated temperatures (e.g., up to about 120° F., or 50° C.) and/or high humidity (e.g., up to; or greater than or equal to about 80% RH), even at high volatile material concentrations. That is, the composition will not change shape or physical state under such conditions. In certain embodiments, the composition provides a structure that will not change its physical state (e.g., become more liquid) even when it absorbs water, such as humidity.

The composition may, in some embodiments, also be advantageous in that it may contain relatively high levels of volatile material (e.g., from about 25% to about 75% by weight of the composition). The composition can also incorporate a large number, range, spectrum (or portfolio) of different volatile materials. This is possible due to the ability to alter/adjust the polarity of the carrier to match the polarity of the volatile material by modifying the level of the structurant (e.g., hydrogenated castor oil). For example, in the case of the compositions described herein, the polarity of the volatile material(s) can be in the range of from about 2 to about 5 Debyes, yet the compositions may still be stable under a wide range of storage conditions. This allows combinations of perfumes that are typically not compatible to be incorporated into compositions (for example, vanilla, coffee, cinnamon, which are very polar, can be combined with fruits (e.g., lemon), or other types of perfume ingredients that are at the other end of the polarity spectrum. In addition, the structure of the composition that incorporates the volatile material(s) may be reversible (that is, it can be converted from a more solid state (e.g, a pliable solid) to a more liquid state, and then back to a more solid state). This may provide the composition with handling, storing, and processability benefits. The term reversible is used with respect to a change in the physical state of the composition and not to the ability to return to its initial condition. It should be understood that the amount of volatile components released or lost during use is an irreversible process.

In a preferred embodiment, the volatile-containing composition of the present invention is contained in a device. Preferably, the device has a window that allows a user of the device to view the composition as it changes color. In addition, the device preferably has a color code or color guide to assist a user of the device in interpreting color changes in the composition.

One embodiment of the present invention provides a method of providing a visual indication of the depletion of a volatile material from a volatile material-containing composition. The method comprises providing a volatile material-containing composition comprising a carrier, a volatile dye that is miscible in the carrier and at least one volatile material that is miscible in the carrier. The composition has a melting temperature that is lower than the melting temperature of the carrier. The composition has a first state when energy is not applied to the composition, and a second energized state when energy is applied to the composition. The volatile material-containing composition is heated to a temperature above that of the melting temperature of the carrier, resulting in a portion of the volatile material and the volatile dye evaporating upon heating. The evaporation of the volatile dye results in a color change in the volatile material-containing composition. Preferably, when heat is no longer applied to the volatile material-containing composition, the composition returns to the first state.

In a preferred embodiment, the evaporation rate of the volatile dye is within about 10% of the evaporation rate of the volatile material. More preferably, the evaporation rate of the volatile dye is within about 8% of the evaporation rate of the volatile material. Even more preferably, the evaporation rate of the volatile dye is within about 5% of the evaporation rate of the volatile material.

### EXAMPLES

Table 1 provides some non-limiting examples of scented compositions that can be made according to the description herein.

TABLE 1

| | | | | | 1,4- |
|---|---|---|---|---|---|
| | | | | | Dimethyl- |
| | | | | | 7-(1- |
| Perfume | | Hydrogenated | | | methylethyl)- |
| % | PEG % | Castor Oil % | Wax % | Azulene | Azulene % |
| 25 | 75 | 0 | 0 | 0.01 | — |
| 50 | 45 | 5 | 0 | 0.02 | — |
| 40 | 30 | 30 | 0 | — | 0.0175 |
| 40 | 40 | 20 | 0 | 0.0175 | — |
| 40 | 48 | 10 | 2 | — | 0.02 |
| 50 | 42 | 5 | 3 | — | 0.02 |
| 25 | 0 | 75 | 0 | 0.05 | 0.05 |

The disclosure of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

While particular embodiments of the subject invention have been described, it will be obvious to those skilled in the art that various changes and modifications of the subject invention can be made without departing from the spirit and scope of the invention. In addition, while the present invention has been described in connection with certain specific embodiments thereof, it is to be understood that this is by way of illustration and not by way of limitation and the scope of the invention is defined by the appended claims which should be construed as broadly as the prior art will permit.

What is claimed is:

1. A volatile material-containing composition comprising:
a) a carrier;
b) at least one volatile material; and
c) at least one volatile dye;
wherein said composition has a first state when energy is not applied to said composition, and a second energized state when energy is applied to said composition, wherein the volatile material and volatile dye are emitted at a first level from said volatile material-containing composition in said first state and the volatile material and volatile dye are emitted from said volatile material-containing composition at a second higher level in said second state; wherein said volatile material-containing composition returns to said first state when energy is no longer applied to said volatile material-containing composition, and wherein said composition releases less than 10 mg/hour of said volatile material and less than 10 μg/hour of said volatile dye at 25° C. and 50% relative humidity (RH).

2. A volatile material-containing composition according to claim 1 wherein said composition releases less than 5 mg/hour of said volatile material and less than 5 μg/hour of said volatile dye at 25° C. and 50% relative humidity (RH).

3. A volatile material-containing composition according to claim 1 wherein said composition releases less than 1 mg/hour of said volatile material and less than 1 μg/hour of said volatile dye at 25° C. and 50% relative humidity (RH).

4. A volatile material-containing composition according to claim 1 wherein said composition has a viscosity between about 100,000 and 1,000,000 Cps.

5. A volatile material-containing composition according to claim 1 wherein said carrier comprises at least one of the following: polyethylene glycol having a weight average molecular weight greater than or equal to about 4,000; hydrogenated castor oil; and fatty acids having a chain length greater than or equal to 14 carbon atoms.

6. A volatile material-containing composition according to claim 1 wherein the first state of said composition is a pliable solid.

7. A volatile material-containing composition according to claim 1 wherein said composition remains a pliable solid under at least one of the following conditions: at temperatures up to about 50° C., and at a humidity greater than or equal to about 80% RH.

8. A volatile material-containing composition according to claim 1 wherein said at least one volatile material is selected from the group consisting of perfume oils, flavors, pesticides, repellants, and mixtures thereof.

9. A volatile material-containing composition according to claim 8 wherein said composition contains up to about 75% of volatile material by weight.

10. A volatile material-containing composition according to claim 1 wherein said at least one volatile dye has a Kovat's index of from about 1000 to about 2000.

11. A volatile material-containing composition according to claim 1 wherein said at least one volatile dye has an azulene base structure.

12. A volatile material-containing composition according to claim 1 wherein said at least one volatile dye is selected from the group consisting of 1,4-Dimethyl-7-(1-methylethyl)-Azulene, Azulene, and mixtures thereof.

13. A volatile material-containing composition according to claim 1 wherein said at least one volatile dye comprises 1,4-Dimethyl-7-(1-methylethyl)-Azulene.

14. A volatile material-containing composition according to claim 1 wherein said at least one volatile dye comprises Azulene.

15. A volatile material-containing composition according to claim 1 wherein said at least one volatile dye comprises a mixture of 1,4-Dimethyl-7-(1-methylethyl)-Azulene and Azulene.

16. A volatile material-containing composition according to claim 11 wherein said composition contains up to about 0.1% of volatile dye by weight.

17. A volatile material-containing composition according to claim 6 wherein said the pliable solid retains at least about 80% of said volatile material at temperatures up to about 50° C. when completely exposed for about 2 weeks.

18. A volatile material-containing composition according to claim 17 wherein said composition can be thermally triggered at temperatures above 50° C. to emit at least some of the volatile material and the volatile dye.

19. A volatile material-containing composition according to claim 1 further comprising a structurant.

20. The volatile material-containing composition of claim 19 wherein the structurant is a substance that comprises a divalent cation.

21. The volatile material-containing composition of claim 20 wherein the structurant is selected from the group consisting of: magnesium and calcium containing molecules such as magnesium and calcium chloride, magnesium and calcium carbonate, and mixtures thereof.

22. The volatile material-containing composition according to claim 1 wherein said composition is melted in said second state.

23. The volatile material-containing composition according to claim 1 wherein said volatile material has a surface tension, and said volatile material-containing composition is placed on a supporting material, and the volatile material-containing composition has a surface tension that is greater than that of the volatile material alone, and the surface tension of the volatile material-containing composition is higher than 20 dyne/cm and lower than 25 dyne/cm.

24. A volatile material-containing composition comprising:
polyethylene glycol comprising between about 25% and about 75%, by weight of said composition;
at least one volatile material, comprising between about 5% and about 75%, by weight of said composition; and
at least one volatile dye, comprising between about 0.001% and about 0.1% by weight of said composition;
wherein said composition is in the form of a pliable solid at room temperature.

25. The volatile material-containing composition of claim 24 wherein said at least one volatile material comprises between about 10 to about 75 weight percent of said composition.

26. A method of providing a visual indication of the depletion of a volatile material from a volatile material-containing composition, said method comprising:
a) providing a volatile material-containing composition comprising a carrier, a volatile dye that is miscible in said carrier and at least one volatile material that is miscible in said carrier, wherein said carrier has a melting temperature, and said composition has a melting temperature that is lower than the melting temperature of said carrier, wherein said composition has a first state when energy is not applied to said composition, and a second energized state when energy is applied to said composition; and
b) heating said volatile material-containing composition to a temperature above that of the melting temperature of said carrier, wherein a portion of said volatile material and said volatile dye are evaporated upon heating, and further, wherein the evaporation of said volatile dye results in a color change in said volatile material-containing composition.

27. The method of claim 26 wherein when heat is no longer applied to said volatile material-containing composition, said composition returns to said first state.

28. The method of claim 26 wherein the evaporation rate of said volatile dye is within about 5% of the evaporation rate of said volatile material.

29. The method of claim 26 wherein the evaporation rate of said volatile dye is within about 10% of the evaporation rate of said volatile material.

30. The method of claim 27 wherein the first state of said composition is a pliable solid.

31. A device containing a volatile material-containing composition capable of a visible determination of its extent of use comprising:
a) a carrier;
b) at least one volatile material; and
c) at least one volatile dye;
wherein said composition changes color as said volatile dye is depleted, and further, wherein said device has a window that allows a user of said device to view said composition as it changes color.

32. The device of claim 31 wherein said composition changes color as an accurate indicator of the depletion of said volatile material.

33. The device of claim 32 wherein said device has a color code or color guide to assist a user of the device in interpreting color changes in said composition.
